(19) United States
(12) Patent Application Publication
(10) Pub. No.: US 2006/0177846 A1
(43) Pub. Date: Aug. 10, 2006

(54) Method and apparatus for design and display of primers

(75) Inventors: Takamune Yamamoto, Tokyo (JP); Noriyuki Yamamoto, Tokyo (JP); Daisuke Sakurai, Tokyo (JP)

Correspondence Address: Reed Smith LLP, Suite 1400, 3110 Fairfax Park Drive, Falls Church, VA, 22042-4503, US

(73) Assignee: Hitachi Software Engineering Co., Ltd.

(21) Appl. No.: 11/289,378

(22) Filed: Nov. 30, 2005

(30) Foreign Application Priority Data
Dec. 7, 2004 (JP) 2004-354063

(51) Int. Cl. C12Q 1/68; G06F 19/00

(52) U.S. Cl. 435/6; 702/20

(57) ABSTRACT

A technology that allows a consensus sequence targeted for primer design to be easily displayed is provided. In sequence data of analysis targets, a consensus nucleotide sequence is generated by performing multiple sequence alignment based on nucleotide sequence, and a consensus amino acid sequence is generated by performing multiple sequence alignment based on amino acid sequence, followed by generating additional consensus nucleotide sequence by reverse translation of the consensus amino acid sequence. In other words, two consensus sequences consisting of the consensus sequence based on nucleotide sequence and the consensus sequence based on amino acid sequence are generated. One of these two consensus sequences can be chosen as a target for primers on a screen to input parameters for primer design.

### FIELD OF THE INVENTION

The present invention relates to a method and an apparatus for design and display of primers for use in a polymerase chain reaction (PCR), and more particularly to a method and an apparatus for design and display of primers that contain degeneracy.

### BACKGROUND OF THE INVENTION

In the field of biology, base sequences of genes from various organisms have been elucidated by genome projects. However, base sequences of genes have not determined for all species of organisms. In the research of species of organisms lagging behind in these projects, a common method is that individual researchers prepare gene libraries and carry out analyses by themselves. When an unknown target gene or a target protein with unknown function is well-defined and a homologous gene (gene having a similar function) from the same species of organism is known or when the research on the target gene or target protein is advanced in similar organisms, these can be compared (multiple sequence alignment), and a region conserved evolutionarily or a region with less variation is extracted, thereby allowing amplification by PCR.

Generally, a function of a protein is exerted by the positional relation and distance of amino acid residues (conformation). In homologous organisms that have undergone a similar evolutionary process, it is highly likely that not only does each functional protein have a similar mechanism but also the kind and conformation of amino acid residues necessary for each function are not altered. Based on this, a method is known in which the amino acid sequence of a protein is compared to that of a known protein, PCR primers are designed from the nucleotide sequence predicted (reverse-translated) from a common sequence (invariable sequence; hereinafter, referred to as consensus sequence), and then an unknown gene is amplified. However, there are only four kinds of bases, while there are 20 different kinds of amino acids; therefore, the correspondence of base and amino acid is not one to one. In fact, when an organism synthesizes a protein from DNA, three bases (codon) correspond to one amino acid (64 to 20). Codons that can be predicted from one amino acid are from one to six kinds and vary depending on each amino acid. This concept is called degeneracy.

As shown in FIG. 11, the bases predicted from one amino acid have several combinations, and therefore, it may not be possible for the whole sequence to be perfectly defined. It is difficult to design primers targeting on the base sequences predicted from the amino acid sequence. However, since there is a bias of codon usage characteristic of organism's species, it is possible to narrow the primer design to some extent by comparing base sequences. When such primers are designed, a method for primer design in which the result of amino acid sequence comparison and the result of nucleotide sequence comparison are judged in a comprehensive manner to design the primers is effective. Although there is a program to design primers based on the results of multiple sequence alignment as an available product (Bioinformatics. Jul. 10, 2004; 20(10): 1644-5), it does not allow to compare the result of nucleotide sequence comparison and the result of amino acid sequence comparison. Therefore, the development of an apparatus that allows easy comparison between a consensus sequence based on amino acid sequence and a consensus sequence based on nucleotide sequence and primer design with ease is desired.

In JP-A No. 210175/2003, a method in which positional information of mutations or polymorphisms in the nucleotide sequence of a gene to be replicated is retrieved, thereby automatically generating information on primers, is disclosed.

### SUMMARY OF THE INVENTION

The functions desired for an apparatus for design and display of primers include the followings: 1. Nucleotide sequences and amino acid sequences of genes can be extracted from common files such as public databases. 2. A consensus nucleotide sequence can be generated by multiple sequence alignment among base sequences. 3. A consensus amino acid sequence can be generated by multiple sequence alignment among amino acid sequences, and then a predicted consensus nucleotide sequence can be generated by its reverse translation. 4. The consensus sequence obtained by the multiple sequence alignment among the base sequences and the consensus sequence obtained by the multiple sequence alignment among the amino acid sequences can be displayed on the same screen for comparison, and a user can compare them. 5. The user can freely choose a target for primer design. 6. Information on designed primers can be clearly displayed.

The functions listed in 1, 2, 3, and 6 can be realized by a conventional apparatus and method. However, an apparatus and a method that satisfy all conditions including the functions 4 and 5 do not exist.

In a method in which a consensus sequence is generated by comparison of base sequences or amino acid sequences of several kinds of known genes having an analogous function and PCR primers targeting on the consensus sequence are designed, the purpose of the present invention is to provide a technology in which the consensus nucleotide sequence derived from multiple sequence alignment based on nucleotide sequence and the consensus nucleotide sequence obtained by reverse translation of the consensus amino acid sequence derived from multiple sequence alignment based on amino acid sequence can be compared and these consensus sequences to be targeted for primer design can be readily displayed.

According to the present invention, in sequence data of analysis targets, a consensus nucleotide sequence is generated by performing multiple sequence alignment based on nucleotide sequence, and a consensus amino acid sequence is generated by performing multiple sequence alignment based on amino acid sequence, followed by generating additional consensus nucleotide sequence by reverse translation of the consensus amino acid sequence. In other words, two consensus base sequences consisting of the consensus sequence based on nucleotide sequence and the consensus sequence based on amino acid sequence are generated. These two consensus sequences are displayed on the same screen.

On a screen to input parameters for primer design, one of these two consensus sequences to be targeted for primer design can be selected.

According to the present invention, not only can the consensus nucleotide sequence derived from multiple sequence alignment based on nucleotide sequence and the consensus nucleotide sequence obtained by reverse translation of the consensus amino acid sequence derived from multiple sequence alignment based on amino acid sequence be compared but also the consensus sequences to be targeted for primer design can be readily displayed.

### BRIEF DESCRIPTION OF THE DRAWINGS

FIG. 1 is a block diagram showing the structure of an apparatus for design and display of primers according to the present invention;

FIG. 2 is an example of a screen that displays sequence data;

FIG. 3 is an example of a screen to input parameters for multiple sequence alignment;

FIG. 4 is an example of a screen that displays the results of multiple sequence alignment based on nucleotide sequence;

FIG. 5 is an example of a screen that displays the results of multiple sequence alignment based on amino acid sequence;

FIG. 6 is an example of a screen to input parameters for primer design;

FIG. 7 is an example of a screen that displays the results of primer design;

FIG. 8 is an example of a screen that displays detail information on the results of the primer design;

FIG. 9 is a diagram showing an outline of consensus sequence generation, primer design, and display processing according to the present invention;

FIG. 10 is a diagram showing processing of multiple sequence alignment; and

FIG. 11 is a schematic representation that shows correspondence between letters of amino acid sequence and letters of nucleotide sequence.

### DETAILED DESCRIPTION OF THE PREFERRED EMBODIMENTS

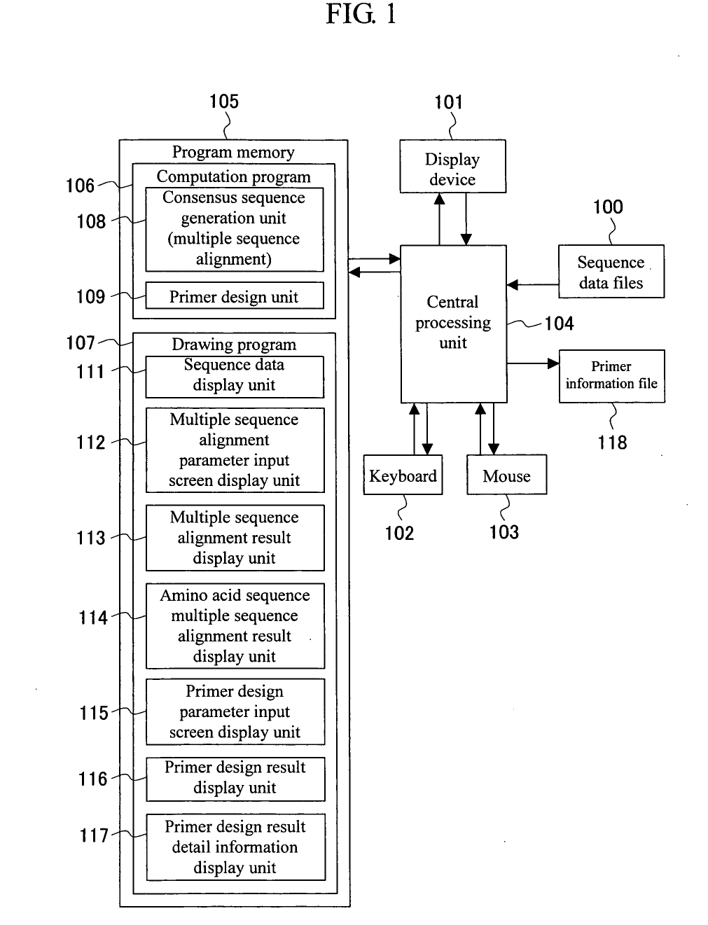

Hereinafter, an embodiment to carry out the present invention is specifically explained with reference to the accompanying drawings. FIG. 1 is a block diagram showing the structure of an apparatus for design and display of primers of the embodiment of the present invention. The apparatus for design and display of primers of the present embodiment is provided with a display device 101 that displays sequence data containing base sequences of genes and amino acid sequences of proteins, results of multiple sequence alignment (consensus sequence), parameters necessary for primer design, and information on designed primers, input units such as a keyboard 102 and a mouse 103 to input information on sequence data, parameters for multiple sequence alignment, and the parameters necessary for primer design, a central processing unit 104, and a program memory 105 that stores programs necessary for processing on the central processing unit 104.

The program memory 105 stores a computation program 106 and a drawing program 107. The computation program 106 contains a consensus sequence generation section 108 to generate consensus sequences according to a multiple sequence alignment method and a primer design section 109 to design primers.

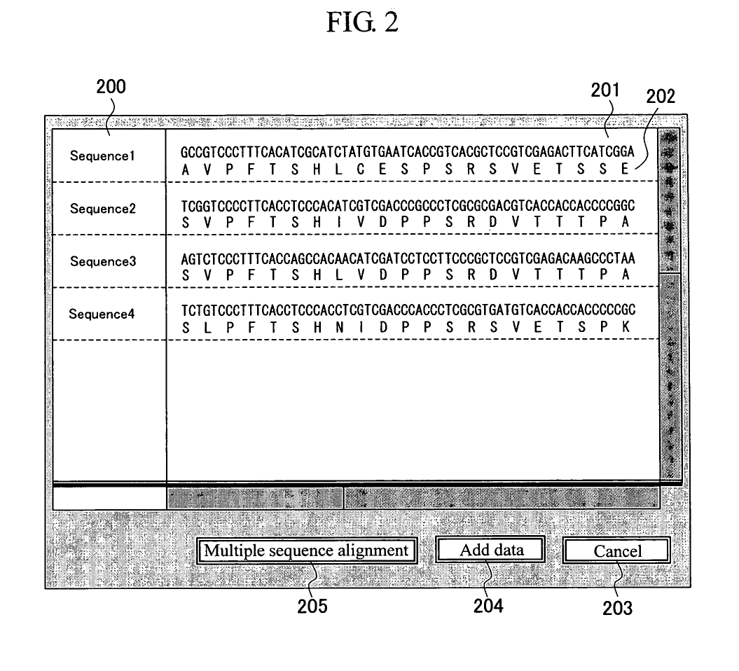

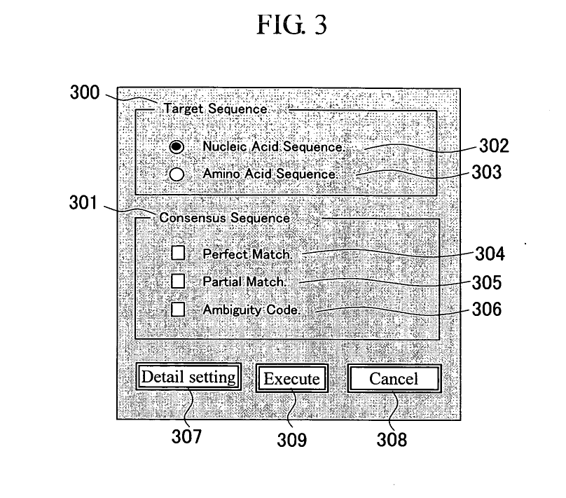

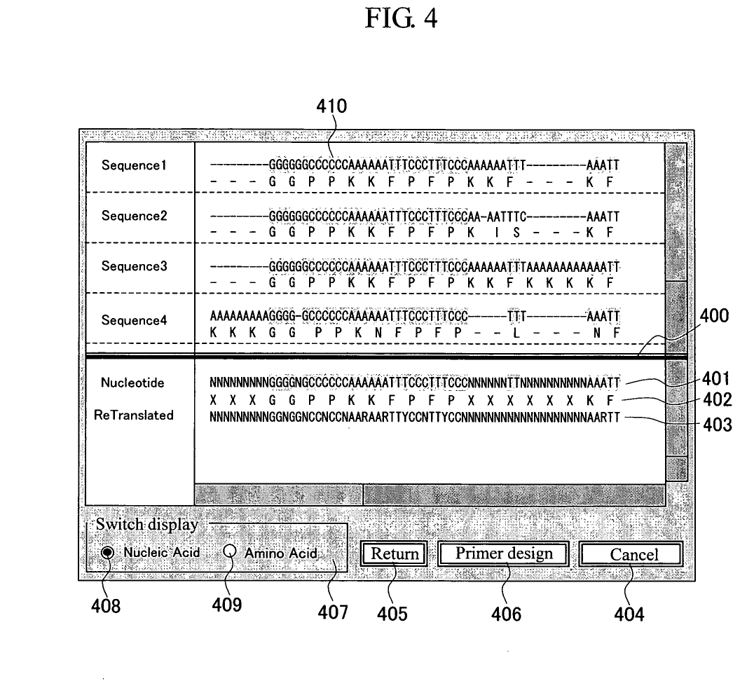

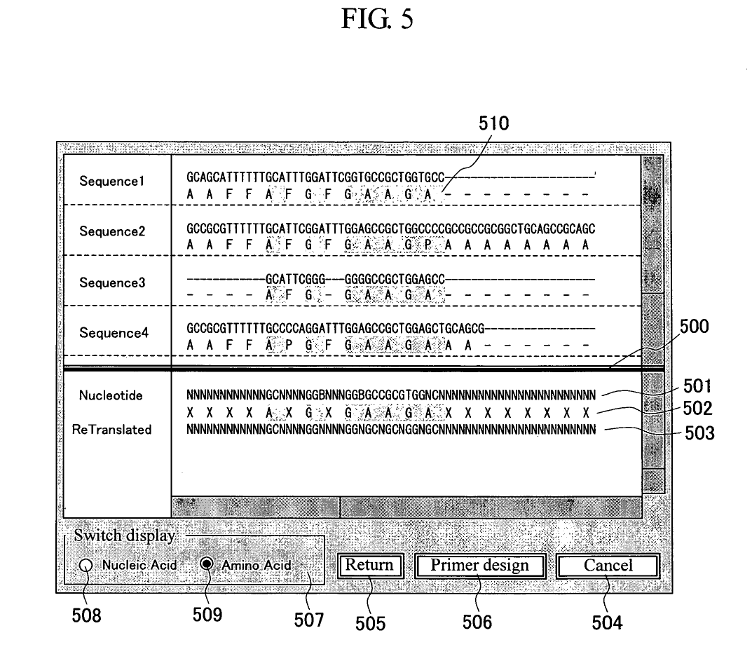

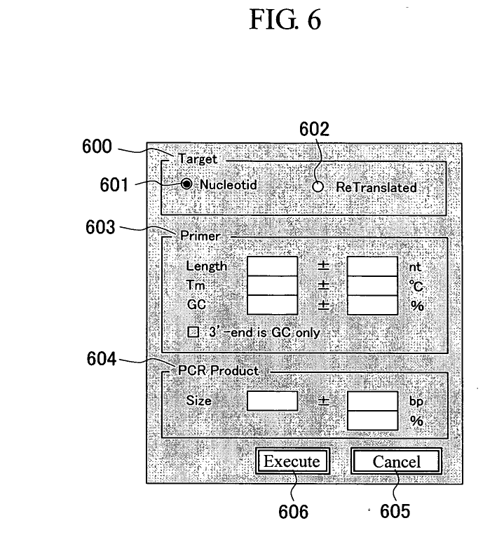

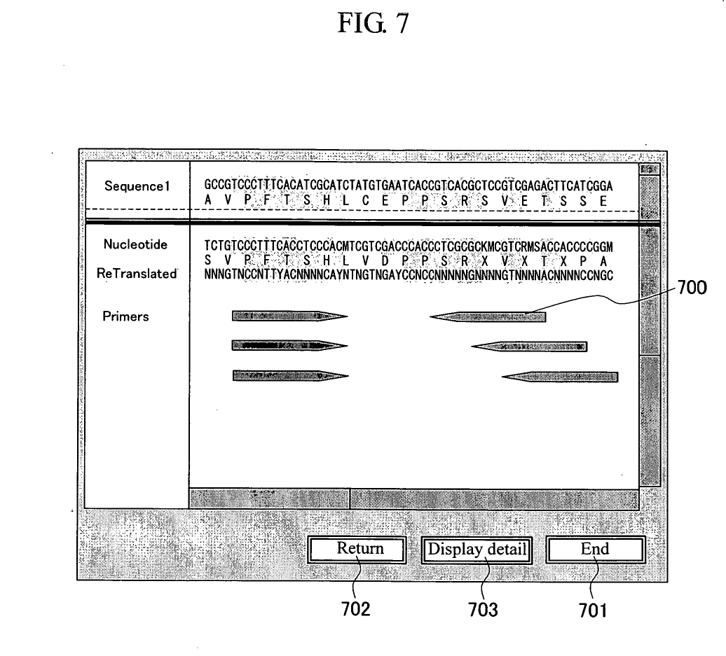

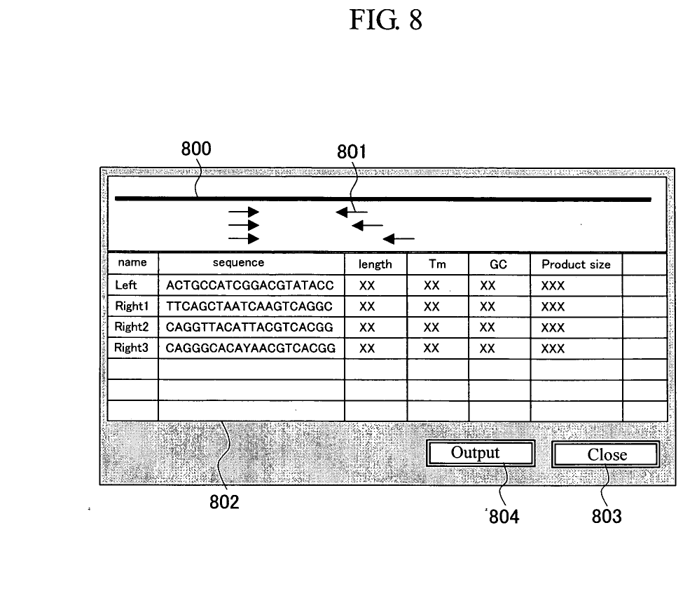

The drawing program 107 is provided with a sequence data display section 111 that draws and displays a screen to display sequence data (FIG. 2), a multiple sequence alignment parameter input screen display section 112 that draws and displays a screen to input parameters for multiple sequence alignment (FIG. 3), a multiple sequence alignment result display section 113 that draws and displays a screen to display the results of multiple sequence alignment based on nucleotide sequence (FIG. 4), an amino acid sequence multiple sequence alignment result display section 114 that draws and displays a screen to display the results of multiple sequence alignment based on amino acid sequence (FIG. 5), a primer design parameter input screen display section 115 that draws and displays a screen to input parameters for primer design (FIG. 6), a primer design result display section 116 that draws and displays a screen to display the results of primer design (FIG. 7), and a primer design result detail information display section 117 that draws and displays a screen to display detail information on the results of primer design (FIG. 8).

The apparatus for design and display of primers retrieves sequence data including base sequences of genes and amino acid sequences from sequence data files 100 such as public databases and stores generated primers in a primer information file 118.

FIG. 2 is an example of the screen to display sequence data including base sequences of genes and amino acid sequences of proteins. This screen is drawn by the sequence data display section 111 of the drawing program 107. This screen has a sequence name 200, a nucleotide sequence 201, an amino acid sequence 202, a button to cancel processing 203, a button to add sequence data 204, and a button to execute multiple sequence alignment 205.

When the apparatus for design and display of primers is activated to start the drawing program 107, this screen is displayed on the display device 101, though the sequence data 200, 201, and 202 are not displayed on the initial screen. In order to display the sequence data, it is necessary to input sequence data. When a user clicks the button to add sequence data 204, a file dialog containing a list of the sequence data files 100 is displayed. When the user selects any one of the sequence data files 100, sequence name, nucleotide sequence, and amino acid sequence are extracted from the selected sequence data file, which are displayed.

With respect to an unknown gene or protein of unknown function that is an analysis target, the user searches for and selects known sequence data of homologous genes or proteins of the same species of organism or homologous organisms. Three letters of nucleotide sequence correspond to one letter of amino acid sequence, and the first letter every three letters of the nucleotide sequence is aligned with a letter of amino acid sequence and displayed. When the input data has information on both sequence and its transcription product, the amino acid sequence is displayed so as to coincide with the coding regions of the nucleotide sequence. For the amino acid sequence corresponding to the noncoding regions and intron portions, “X” is displayed for every three bases. Sequence data is repeatedly added, and a new sequence data is displayed under the preceding sequence data that has already been input.

The button to execute multiple sequence alignment 205 is effective when two or more sequence data are displayed on this screen. Clicking the button 205 allows display of the screen to input parameters for multiple sequence alignment (FIG. 3).

FIG. 3 is an example of the screen to input parameters for multiple sequence alignment. This screen is drawn by the multiple sequence alignment parameter input screen display section 112 of the drawing program 107. This screen is provided with a group to select target sequence for analysis 300, a group to designate condition or method for generating consensus sequence 301, a button to set detail parameters for multiple sequence alignment 307, a button to cancel processing 308, and a button to execute multiple sequence alignment 309. The group to select target sequence for analysis 300 has a radio button to designate nucleotide sequence 302 and a radio button to designate amino acid sequence 303. The group to designate condition or method for generating consensus sequence 301 has a radio button to designate generation of consensus sequence with perfect matching letters 304, a radio button to designate generation of consensus sequence with partial matching letters based on majority rule 305, and a radio button to designate generation of consensus sequence with ambiguity codes 306, where one of the three buttons can be selected. When amino acid sequence is designated as the target sequence for analysis, the radio button to designate generation of consensus sequence with ambiguity codes 306 is disabled and cannot be selected.

When necessary, first the button 307 is clicked, and detail parameters for multiple sequence alignment are set. The radio button 302 or the radio button 303 in the group 300 is selected, and one button is chosen from the group 301. When the button to execute multiple sequence alignment 309 is clicked next, the consensus sequence generation section 108 is run. Multiple sequence alignment based on nucleotide sequence and multiple sequence alignment based on amino acid sequence are performed. When the radio button 302 is selected, the results of the multiple sequence alignment based on nucleotide sequence are displayed on the screen shown in FIG. 4. When the radio button 303 is selected, the results of the multiple sequence alignment based on amino acid sequence are displayed on the screen shown in FIG. 5. When the button 309 is clicked after selecting the radio button 302, multiple sequence alignment based on nucleotide sequence is performed, and the results are displayed on the screen shown in FIG. 4. When the button 309 is clicked after selecting the radio button 303, multiple sequence alignment based on amino acid sequence is performed, and the results are displayed on the screen shown in FIG. 5.

FIG. 4 is an example of the screen showing the results of multiple sequence alignment based on nucleotide sequence. This screen is drawn by the multiple sequence alignment result display section 113 of the drawing program 107. This screen is divided into upper and lower parts by a splitter 400, where the sequence data on the screen in FIG. 2 is displayed in the upper part and a consensus nucleotide sequence 401 resulting from multiple sequence alignment based on nucleotide sequence is displayed in the lower part. Note that a consensus amino acid sequence 402 resulting from multiple sequence alignment based on amino acid sequence and a nucleotide sequence obtained by reverse translation of the consensus amino acid sequence 403 are displayed on this screen at the same time. The nucleotide sequence obtained by the reverse translation is a nucleotide sequence predicted from the consensus amino acid sequence. The letters matching between the base sequences in the upper sequence data and the consensus nucleotide sequence 401 in the lower part are highlighted 410.

This screen is further provided with a button to cancel processing 404, a button to return to previous screen 405, a button to execute primer design 406, and a group to switch display 407.

The group to switch display 407 has a radio button to display results of multiple sequence alignment based on nucleotide sequence 408 and a radio button to display results of multiple sequence alignment based on amino acid sequence 409. On the screen in FIG. 4, the radio button 408 is selected, and therefore, the screen to display the results of multiple sequence alignment based on nucleotide sequence is displayed. When the radio button 409 is selected, the screen to display the results of multiple sequence alignment based on amino acid sequence (FIG. 5) is displayed.

Since both of the consensus sequence based on nucleotide sequence 401 and the consensus sequence based on amino acid sequence 403 are displayed in the lower part of the screen in this example, the user can compare the both sequences. The user can choose for which sequence primers should be designed by comparing the both sequences. When primers are designed for the consensus sequence based on nucleotide sequence 401, the button to execute primer design 406 is clicked, thereby displaying the screen to input parameters for primer design (FIG. 6). When the button to execute primer design 406 is clicked under the condition that one or more bases of the consensus sequence are selected, primers are designed so that the PCR amplification product may contain the selected region. When not selected, primers are designed by targeting on the entire consensus sequence.

When primers are designed for the consensus sequence based on amino acid sequence 403, the radio button 409 is clicked, thereby displaying the screen to display the results of multiple sequence alignment based on amino acid sequence (FIG. 5).

FIG. 5 is an example of the screen showing the results of multiple sequence alignment based on amino acid sequence. This screen is drawn by the amino acid sequence multiple sequence alignment result display section 114 of the drawing program 107. This screen is divided into upper and lower parts by a splitter 500, where the sequence data on the screen in FIG. 2 is displayed in the upper part and a consensus amino acid sequence 502 resulting from multiple sequence alignment based on amino acid sequence and a nucleotide sequence obtained by reverse translation of the consensus amino acid sequence 503 are displayed in the lower part. The nucleotide sequence obtained by reverse translation is a nucleotide sequence predicted from the consensus amino acid sequence. Note that a consensus nucleotide sequence resulting from multiple sequence alignment based on nucleotide sequence is also displayed on this screen at the same time. The letters matching between the amino acid sequences in the upper sequence data and the consensus amino acid sequence 502 in the lower part are highlighted 510.

This screen is further provided with a button to cancel processing 504, a button to return to previous screen 505, a button to execute primer design 506, and a group to switch display 507. The functions of these buttons 504, 505, and 506 and the group 507 are the same as those of the buttons 404, 405, and 406 and the group 407 on the screen in FIG. 4.

FIG. 6 is an example of the screen to input parameters for primer design. This screen is drawn by the primer design parameter input screen display section 115 of the drawing program 107. The screen of this example is provided with a group to designate consensus sequence 600 that is the target for primer design, a group to input primer conditions 603, a group to input conditions for PCR product 604, a button to cancel processing 605, and a button to execute processing 606. The group to designate consensus sequence 600 that is the target for primer design has a radio button to select consensus sequence based on nucleotide sequence 601 and a radio button to select nucleotide sequence obtained by reverse translation of consensus sequence based on amino acid sequence 602.

When the consensus sequence shown in FIG. 4 that has resulted from multiple sequence alignment based on nucleotide sequence is selected as a target for primer design, the radio button 601 is clicked. When the consensus sequence shown in FIG. 5 that has resulted from multiple sequence alignment based on amino acid sequence is selected, the radio button 602 is clicked.

When one of the two radio buttons 601 and 602 is selected and the button to execute processing 606 is clicked, the primer design section 109 is run. The primer design section 109 performs primer design processing for the consensus sequence designated by the user according to the input condition or method. The results of primer design are displayed on the screen in FIG. 7.

FIG. 7 is an example of the screen that displays the results of primer design. This screen is drawn by the primer design result display section 116 of the drawing program 107. This screen is divided into upper and lower parts by a splitter, where the sequence data in FIG. 2 is displayed in the upper part, and a consensus sequence that is the target of primer design and arrows to indicate positional information of primers 700 are displayed in the lower part. The arrows 700 are arranged at the positions corresponding to the consensus sequence. The primers are generally treated as one primer set consisting of a primer for the sense strand and a plurality of primers for the nonsense strand. Hereinafter, the one primer set is simply referred to as primer.

This screen is further provided with a button to end processing 701, a button to return to previous screen 702, and a button to display detail information on the results of primer design 703. When the button to display detail information 703 is clicked under the state that the arrows to indicate positional information of primers 700 are selected, the screen to display detail information on the results of primer design (FIG. 8) is displayed. When the button to end processing 701 is clicked, the processing is terminated, and all windows are closed.

FIG. 8 is an example of the screen to display detail information on the results of primer design. This screen is drawn by the primer design result detail information display section 117 of the drawing program 107. This screen is provided with a consensus sequence that is the target of primer design 800, arrows to indicate positional information of primers 801, detail information on primers 802, a button to close window 803, and a button to output results 804.

The size of the consensus sequence that is the target of primer design 800 is controlled such that the whole sequence is displayed within the window. The arrows to indicate positional information of primers 801 are arranged at the positions corresponding to the consensus sequence that is the target of primer design. The arrows to indicate positional information of primers 801 are displayed only for a primer that has been selected on the screen in FIG. 7. When another primer is selected on the screen in FIG. 7, information on the selected primer is displayed. When no primer is selected on the screen in FIG. 7, information on all primers is displayed. When the output button 804 is clicked, information on the primer being displayed is output as a file 118 delimited with tabs. When the button to close window 803 is clicked, the window is closed to return to the screen in FIG. 7.

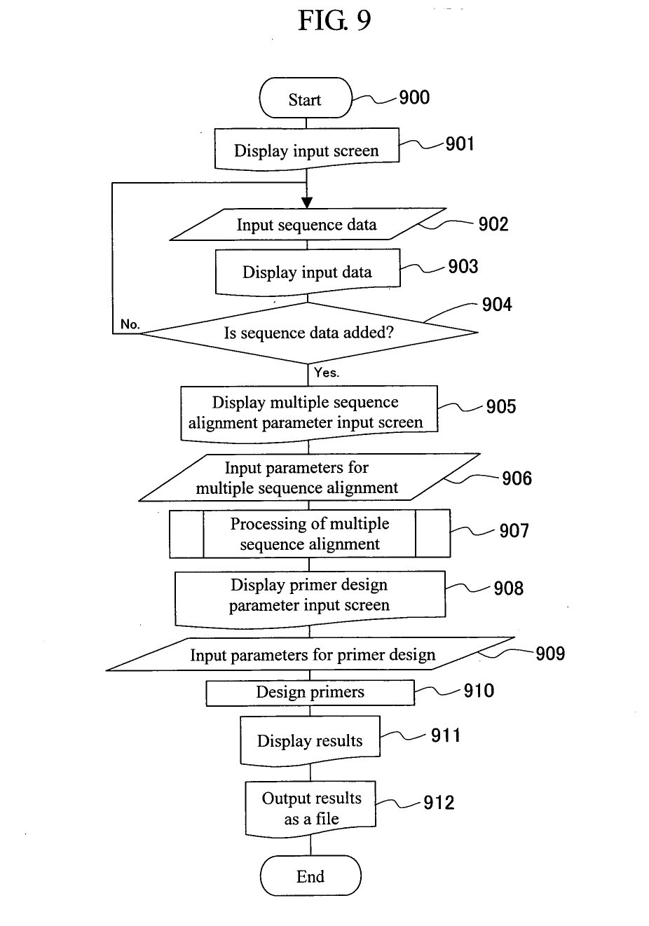

An outline of the consensus sequence generation, primer design, and display processing according to the present invention is explained with reference to FIG. 9. In step 900, the drawing program 107 is activated, and then the sequence data display section 111 that draws and displays the screen to display sequence data (FIG. 2) is started, thereby displaying the screen shown in FIG. 2 on the display device 101 in step 901. In step 902, sequence data is input. When a user clicks the button to add sequence data 204 on the screen in FIG. 2, a file dialog containing a list of the sequence data files 100 is displayed. The user selects any one of the sequence data files 100. In step 903, sequence data is extracted from the selected sequence data file and displayed at the sequence data 201 and 202 on the screen to display sequence data (FIG. 2). In step 904, the user again clicks the button to add sequence data 204 when sequence data is added. In this case, the processes of the steps 902 and 903 are repeated. When sequence data is not added, the user clicks the button to execute multiple sequence alignment 205, thereby advancing to step 905 to activate the multiple sequence alignment parameter input screen display section 112 that draws and displays the screen to input parameters for multiple sequence alignment (FIG. 3).

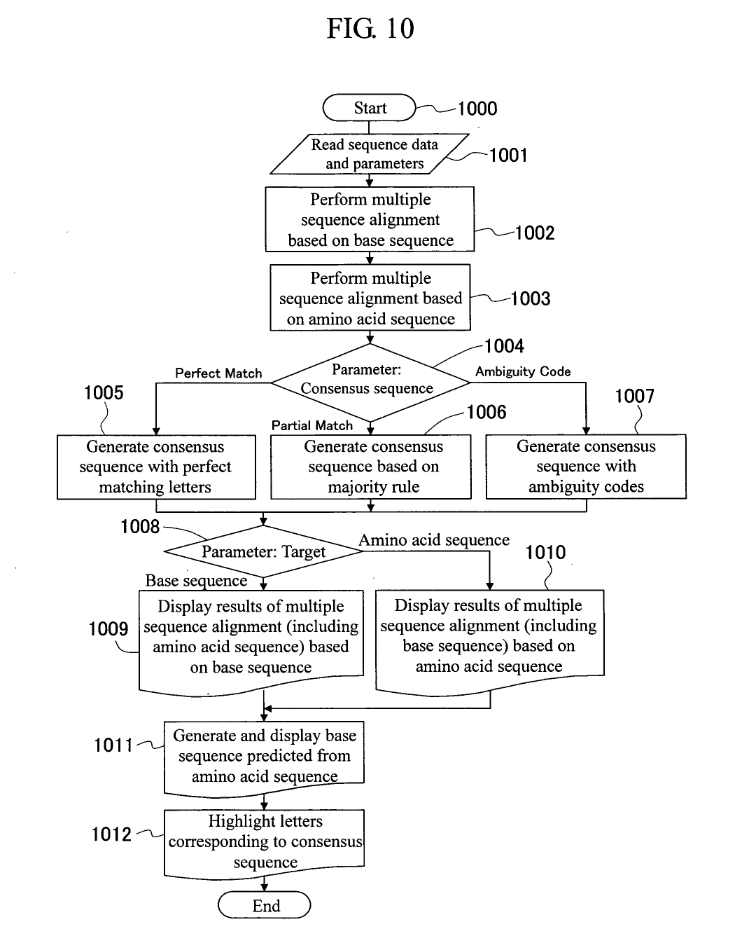

In step 905, the screen shown in FIG. 3 is displayed. In step 906, parameters for multiple sequence alignment are input on the screen in FIG. 3. The parameters include a target sequence for analysis, a condition or method for generating consensus sequence, and the like. In step 907, processing of multiple sequence alignment is performed according to the input parameters. The processing of multiple sequence alignment is executed by the consensus sequence generation section 108, and the results are displayed. The details will be described with reference to FIG. 10. When the user clicks the primer design button 406, the screen shown in FIG. 6 is displayed in step 908.

In step 909, the user inputs parameters for primer design on the screen in FIG. 6. In step 910, the primer design section 109 retrieves sequences suitable for primers based on the input parameters. In step 911, the screen shown in FIG. 7 is displayed, and the results of the primer design are displayed.

When the user clicks the button to display detail information 703, the screen shown in FIG. 8 is displayed, and the results are output as a file in step 912. In the present example, a primer containing degeneracy can be obtained.

The processing of multiple sequence alignment is explained with reference to FIG. 10. This processing represents the processing in the step 907 of FIG. 9 and is executed by the consensus sequence generation section 108. In step 1000, the consensus sequence generation section 108 is activated, and in step 1001, a plurality of sequence data that are the analysis targets and the parameters are read. In step 1002, multiple sequence alignment is performed for the input base sequences. In step 1003, multiple sequence alignment is performed for the input amino acid sequences. In step 1004, the condition or method designated as the parameter is judged.

In the case where “Perfect Match” is selected, consensus sequence is identified only when the result of the multiple sequence alignment shows that sequences at an equivalent position have all identical letters; otherwise “N” is used in step 1005. In the case where “Partial Match” is selected, the largest number of a letter at an equivalent position resulting from multiple sequence alignment is identified as the consensus sequence in step 1006, and when the numbers of letters are the same, “N” is used. In the case where “Ambiguity Code” is selected, a consensus sequence is generated from sequences at an equivalent position in the result of multiple sequence alignment using ambiguity codes in step 1007.

In step 1008, the sequence data of the analysis targets and the parameters that have been input in FIG. 3 are read.

When the target for analysis is nucleotide sequence (302), consensus base sequences 401 and 501 that are the results of multiple sequence alignment based on nucleotide sequence are displayed in step 1009.

When the target for analysis is amino acid sequence (303), consensus amino acid sequences 402 and 502 that are the results of multiple sequence alignment based on amino acid sequence are displayed in step 1010.

In step 1011, consensus base sequences 403 and 503 that have been obtained by reverse translation of the consensus amino acid sequences 402 and 502 respectively are displayed. In step 1012, letters corresponding to consensus sequences are highlighted (410 and 510).

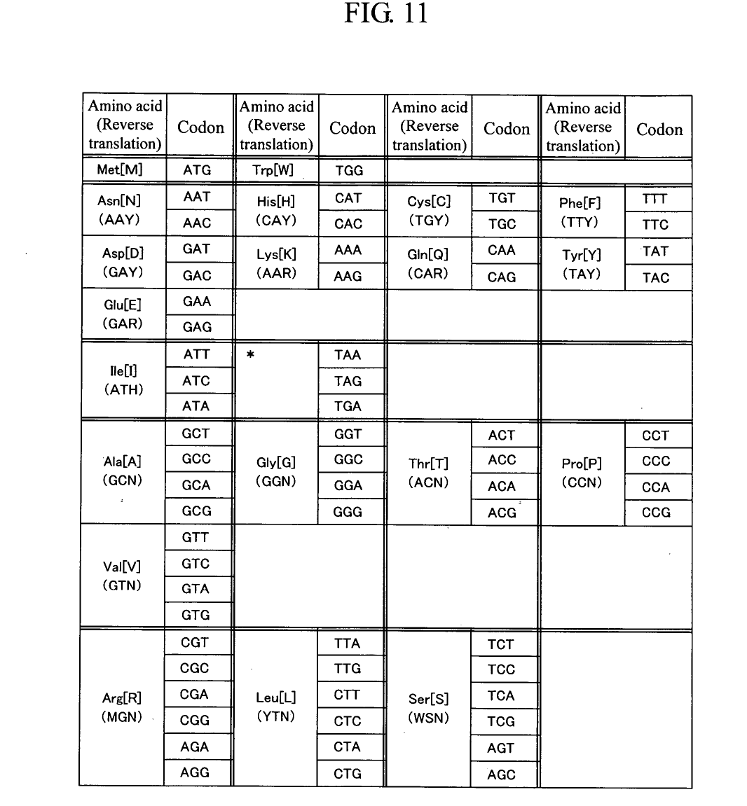

FIG. 11 is a schematic representation to explain correspondence between letters showing amino acid sequence and letters showing nucleotide sequence. In this figure, three-letter abbreviations of amino acids and their corresponding codons (three letters of bases) are shown. One-letter designations for amino acids are shown in square brackets. Nucleotide sequence obtained by reverse translation of amino acid sequence is shown in round brackets using ambiguity codes in consideration of codon degeneracy.

In the foregoing, an embodiment of the present invention has been explained. However, the present invention is not limited to the above embodiment, and it should be understood that various modifications are apparent to one of ordinary skill in the art. Such modifications can be made without departing from the scope of the invention set forth in the appended claims.

What is claimed is:

1. A program for design and display of primers and readable by a computer to perform the steps of:
retrieving sequence data of analysis targets;
inputting conditions for multiple sequence alignment via an input unit;
generating a first consensus nucleotide sequence by executing multiple sequence alignment based on nucleotide sequence for the sequence data of analysis targets according to the conditions for multiple sequence alignment via a computing unit;
generating a second consensus nucleotide sequence by means of generating a consensus amino acid sequence by executing multiple sequence alignment based on amino acid sequence for the sequence data of analysis targets according to the conditions for multiple sequence alignment, followed by reverse translation of the consensus amino acid sequence, via the computing unit;
displaying the first consensus nucleotide sequence and the second consensus nucleotide sequence on the same screen via a display unit;
inputting parameters for primer design via the input unit;
performing primer design for the first or the second consensus nucleotide sequence according to the parameters for primer design via the computing unit; and
displaying the results of primer design via the display unit.

2. The program for design and display of primers according to claim 1, wherein the step of displaying the consensus sequences further comprises the steps of;
displaying a first consensus sequence screen that displays not only the first consensus nucleotide sequence and base sequences of the sequence data of analysis targets with common letters highlighted but also the second consensus nucleotide sequence via the display unit; and
displaying a second consensus sequence screen that displays not only the consensus amino acid sequence and amino acid sequences of the sequence data of analysis targets with common letters highlighted but also the first consensus nucleotide sequence via the display unit;
wherein each of the first consensus sequence screen and the second consensus sequence screen can be displayed in a switchable manner according to an input command via the input unit.

3. The program for design and display of primers according to claim 1, wherein the step of retrieving the sequence data of analysis targets further comprises the steps of;
reading the sequence data of analysis targets from sequence data files stored in existing databases; and
displaying sequence names, base sequences of genes, and amino acid sequences of proteins via the display unit.

4. The program for design and display of primers according to claim 1, wherein the step of inputting conditions for multiple sequence alignment further comprises the steps of:
displaying a screen to input parameters for multiple sequence alignment via the display unit; and
inputting parameters for multiple sequence alignment that are selected on the screen to input parameters for multiple sequence alignment via the input unit.

5. The program for design and display of primers according to claim 1, wherein the step of inputting parameters for primer design further comprises the steps of:
displaying a screen to input conditions for primer design that contains display to select one of the two consensus base sequences as a target for primer design and display to input conditions for primer design via the display unit; and
inputting the conditions for primer design that are selected on the screen to input conditions for primer design via the input unit.

6. The program for design and display of primers according to claim 1, wherein the step of displaying the results of primer design further comprises:
displaying the sequence data of analysis targets and primers on the same screen via the display unit such that the primers are arranged at positions corresponding to the sequence data.

7. The program for design and display of primers according to claim 1, wherein detail information containing the nucleotide sequence of selected primers is further displayed via the display unit when one of the results of the primer design is selected via the input unit.

8. An apparatus for design and display of primers provided with an input unit to input data and a command, a program memory to store a program, a central processing unit to execute the program, and a display device to display designed primers,
the program containing a consensus sequence generation unit to generate a consensus sequence by a multiple sequence alignment method and a primer design unit to design primers,
the consensus sequence generation unit generating a first consensus nucleotide sequence by executing multiple sequence alignment based on nucleotide sequence according to sequence data of analysis targets and conditions for multiple sequence alignment that are input by the input unit as well as a second consensus nucleotide sequence by means of generating a consensus amino acid sequence by executing multiple sequence alignment based on amino acid sequence, followed by reverse translation of the consensus amino acid sequence, and
the display device displaying a screen that contains the two consensus base sequences.

9. The apparatus for design and display of primers according to claim 8, wherein the display device displays, in a switchable manner according to an input by the input unit, a first consensus sequence screen that displays not only the first consensus nucleotide sequence and base sequences of the sequence data of analysis targets with common letters highlighted but also the second consensus nucleotide sequence and a second consensus sequence screen that displays not only the consensus amino acid sequence and amino acid sequences of the sequence data of analysis targets with common letters highlighted but also the first consensus nucleotide sequence.

10. The apparatus for design and display of primers according to claim 8, wherein the primer design unit designs primers for one of the two consensus base sequences according to conditions for primer design that are input by the input unit and displays the designed primers on the display device.

11. The apparatus for design and display of primers according to claim 8, wherein the program includes a program to draw and display a screen that displays sequence data, a program to draw and display a screen that displays the results of multiple sequence alignment based on nucleotide sequence for the sequence data, a program to draw and display a screen that displays the results of multiple sequence alignment based on amino acid sequence for the sequence data, a program to draw and display a screen to input parameters for primer design, and a program to draw and display a screen that displays the results of primer design.

12. The apparatus for design and display of primers according to claim 11, wherein the program further includes a program to draw and display a screen to input parameters for multiple sequence alignment, a program to draw and display a screen to input parameters for primer design, and a program to draw and display a screen that displays detail information on the results of primer design.


SEQUENCE LISTING (24 sequences)

SEQ ID NO: 1 - DNA, 62 bp, Artificial Sequence
  Description of Artificial Sequence:Synthetic DNA
gccgtccctttcacatcgcatctatgtgaatcaccgtcacgctccgtcgagacttcatcgga

SEQ ID NO: 2 - DNA, 62 bp, Artificial Sequence
  Description of Artificial Sequence:Synthetic DNA
tcggtccccttcacctcccacatcgtcgacccgccctcgcgcgacgtcaccaccaccccggc

SEQ ID NO: 3 - DNA, 62 bp, Artificial Sequence
  Description of Artificial Sequence:Synthetic DNA
agtctccctttcaccagccacaacatcgatcctccttcccgctccgtcgagacaagccctaa

SEQ ID NO: 4 - DNA, 62 bp, Artificial Sequence
  Description of Artificial Sequence:Synthetic DNA
tctgtccctttcacctcccacctcgtcgacccaccctcgcgtgatgtcaccaccacccccgc

SEQ ID NO: 5 - DNA, 39 bp, Artificial Sequence
  Description of Artificial Sequence:Synthetic DNA
ggggggccccccaaaaaatttccctttcccaaaaaattt

SEQ ID NO: 6 - DNA, 32 bp, Artificial Sequence
  Description of Artificial Sequence:Synthetic DNA
ggggggccccccaaaaaatttccctttcccaa

SEQ ID NO: 7 - DNA, 53 bp, Artificial Sequence
  Description of Artificial Sequence:Synthetic DNA
ggggggccccccaaaaaatttccctttcccaaaaaatttaaaaaaaaaaaatt

SEQ ID NO: 8 - DNA, 13 bp, Artificial Sequence
  Description of Artificial Sequence:Synthetic DNA
aaaaaaaaagggg

SEQ ID NO: 9 - DNA, 25 bp, Artificial Sequence
  Description of Artificial Sequence:Synthetic DNA
gccccccaaaaaatttccctttccc

SEQ ID NO: 10 - DNA, 62 bp, Artificial Sequence
  misc_feature (1)...(9), (14), (40)...(45), (48)...(57): Synthetic DNA, n is a, c, g or t
nnnnnnnnnggggngccccccaaaaaatttccctttcccnnnnnnttnnnnnnnnnnaaatt

SEQ ID NO: 11 - DNA, 62 bp, Artificial Sequence
  misc_feature (1)...(9), (12), (15), (18), (21), (33), (39)...(57): Synthetic DNA, n is a, c, g or t
nnnnnnnnnggnggnccnccnaaraarttyccnttyccnnnnnnnnnnnnnnnnnnnaartt

SEQ ID NO: 12 - DNA, 39 bp, Artificial Sequence
  Description of Artificial Sequence:Synthetic DNA
gcagcattttttgcatttggattcggtgccgctggtgcc

SEQ ID NO: 13 - DNA, 62 bp, Artificial Sequence
  Description of Artificial Sequence:Synthetic DNA
gccgcgttttttgcattcggatttggagccgctggccccgccgccgcggctgcagccgcagc

SEQ ID NO: 14 - DNA, 15 bp, Artificial Sequence
  Description of Artificial Sequence:Synthetic DNA
ggggccgctggagcc

SEQ ID NO: 15 - DNA, 45 bp, Artificial Sequence
  Description of Artificial Sequence:Synthetic DNA
gccgcgttttttgccccaggatttggagccgctggagctgcagcg

SEQ ID NO: 16 - DNA, 62 bp, Artificial Sequence
  misc_feature (1)...(12), (15)...(18), (22)...(24), (37), (39)...(62): Synthetic DNA, n is a, c, g or t
nnnnnnnnnnnngcnnnnggbnnnggbgccgcgtggncnnnnnnnnnnnnnnnnnnnnnnnn

SEQ ID NO: 17 - DNA, 62 bp, Artificial Sequence
  misc_feature (1)...(12),(15)...(18),(21)...(24),(27),(30),(33),(36),(39)...(62): Synthetic DNA, n is a, c, g or t
nnnnnnnnnnnngcnnnnggnnnnggngcngcnggngcnnnnnnnnnnnnnnnnnnnnnnnn

SEQ ID NO: 18 - DNA, 62 bp, Artificial Sequence; identical to SEQ ID NO: 1
  Description of Artificial Sequence:Synthetic DNA

SEQ ID NO: 19 - DNA, 62 bp, Artificial Sequence
  Description of Artificial Sequence:Synthetic DNA
tctgtccctttcacctcccacmtcgtcgacccaccctcgcgckmcgtcrmsaccaccccggm

SEQ ID NO: 20 - DNA, 62 bp, Artificial Sequence
  misc_feature (1)...(3),(6),(9),(15)...(18),(22),(24),(27),(33),(36)...(40),(42)...(45),(48)...(51), (54)...(57), (60): Synthetic DNA, n is a, c, g or t
nnngtnccnttyacnnnncayntngtngayccnccnnnnngnnnngtnnnnacnnnnccngc

SEQ ID NO: 21 - DNA, 20 bp, Artificial Sequence
  Description of Artificial Sequence:Synthetic DNA
actgccatcggacgtatacc

SEQ ID NO: 22 - DNA, 20 bp, Artificial Sequence
  Description of Artificial Sequence:Synthetic DNA
ttcagctaatcaagtcaggc

SEQ ID NO: 23 - DNA, 20 bp, Artificial Sequence
  Description of Artificial Sequence:Synthetic DNA
caggttacattacgtcacgg

SEQ ID NO: 24 - DNA, 20 bp, Artificial Sequence
  Description of Artificial Sequence:Synthetic DNA
cagggcacayaacgtcacgg